(19) United States
(12) Patent Application Publication
(10) Pub. No.: US 2022/0290248 A1
(43) Pub. Date: Sep. 15, 2022

(54) SYSTEM AND METHOD FOR ASSESSING THE RISK OF COLORECTAL CANCER

(71) Applicant: Tata Consultancy Services Limited, Mumbai (IN)

(72) Inventors: SHARMILA SHEKHAR MANDE, Pune (IN); TUNGADRI BOSE, Pune (IN); SUBHRAJIT BHAR, Pune (IN); ANIRBAN DUTTA, Pune (IN); RASHMI SINGH, Pune (IN)

(73) Assignee: Tata Consultancy Services Limited, Mumbai (IN)

(21) Appl. No.: 17/634,949

(22) PCT Filed: Aug. 12, 2020
(86) PCT No.: PCT/IB2020/057585

§ 371 Date: Feb. 11, 2022

(30) Foreign Application Priority Data
Aug. 13, 2019 (IN) 201921032793

(51) Int. Cl. C12Q 1/6886; C12Q 1/689

(52) U.S. Cl. CPC C12Q 1/6886; C12Q 1/689

(57) ABSTRACT

Colorectal cancer is a severe disease, if not assessed properly, it may lead to the death of an individual. A system and method for assessing the risk of colorectal cancer has been provided. The system is configured to assess individuals to check the risk of presence of colorectal cancer (CRC) and/or adenomatous (colonic/rectal) polyps, by quantifying the abundance of sensory proteins in their gut microbiome. The system further categorizes the person into one of healthy, adenoma and cancerous categories based on the nature and abundance of sensory proteins in the gut microbiome. The system further describes microbiota based therapeutics for treatment of the person with colorectal adenoma and/or cancer through administration of at least one of a consortium of healthy microbes, antibiotic drugs and pre-/pro-/syn-/post-biotic compounds or fecal microbiome transplant which could modulate the disease microbiome composition towards a healthy equilibrium.

### CROSS-REFERENCE TO RELATED APPLICATIONS AND PRIORITY

The present application claims priority from Indian provisional application no. 201921032793, filed on Aug. 13, 2019. The entire contents of the aforementioned application are incorporated herein by reference.

### TECHNICAL FIELD

The embodiments herein generally relates to the field of colorectal cancer, and, more particularly, to a method and system for assessing the risk of colorectal cancer in a person.

### BACKGROUND

Every year almost 1.5 million people are diagnosed with colorectal cancer (CRC). CRC is treatable with more than 90% of survival rate if detected at an early stage. But the chances of survival are less than 15% for patients who are detected with advanced stages of cancer. Therefore, it is extremely important to detect the CRC as early as possible. However, there are several challenges associated with the early detection of CRC using the existing CRC assessment techniques.

Currently, colonoscopy and sigmoidoscopy are the most widely used techniques for diagnosis of CRC. Both these diagnostic techniques are invasive in nature and thus the patients have to suffer both physiological and psychological stress to undergo these tests. More recently, computed tomography based colonoscopy procedures have been developed. This procedure, although minimally invasive (only a single probe/scope is inserted for blowing air into the colon and rectum for better visualization), still requires bowel preparation as well as administration of barium enema. Further, all the above mentioned diagnostic procedures for CRC are quite expensive. Moreover, while invasive procedures like colonoscopy and sigmoidoscopy fail to detect any anomaly in certain regions of the colon and rectum (called ‘Blind Spots’) or in cases of poor bowel preparation, the minimally invasive procedures like CT colonoscopy cannot detect polyps of dimensions smaller than 8 mm.

Recently, several biochemical tests with the potential to diagnose CRC have been proposed. These biochemical tests usually measure the altered amount of certain proteins and/or DNA modifications in blood (either directly drawn from the body or that detected in stool). Further, certain biochemical tests teach the use of some metabolites and/or volatile organic compounds in human body as potential markers of CRC. While most of these tests suffer from low sensitivity and/or high false positive rates, the relatively accurate ones are quite expensive to be employed for regular screening of the masses.

A few studies have also suggested the use of microbiome as indicators of CRC. Most of these studies could only identify microbiome based signals that could be used to distinguish between healthy subjects and patients with CRC at a population level. These microbiome signatures are not applicable for disease diagnostics/prognostics for individual subjects.

### SUMMARY

Embodiments of the present disclosure present technological improvements as solutions to one or more of the above-mentioned technical problems recognized by the inventors in conventional systems. For example, in one embodiment, a system for assessing the risk of colorectal cancer in a person has been provided. The system comprises a sample collection module, a DNA extractor, a sequencer, a database creation module, one or more hardware processors and a memory. The sample collection module collects a microbiome sample from gut of the person for the assessment of the risk of CRC, wherein the microbiome sample comprising microbial cells. The DNA extractor extracts DNA from the microbial cells. The sequencer sequences the extracted DNA to get sequenced metagenomic reads. The database creation module creates a database of sensory protein sequences of a plurality of organisms, wherein the database of sensory protein sequences comprises information pertaining to the sensory proteins of all fully or partially sequenced bacterial genomes obtained from a plurality of public repositories. The memory in communication with the one or more hardware processors, wherein the one or more first hardware processors are configured to execute programmed instructions stored in the memory, to: generate sensory protein abundance profiles of a set of control versus adenoma samples, a set of control versus carcinoma samples, and a set of adenoma versus carcinoma samples obtained from publicly available data; apply a random forest classifier on the generated sensory protein abundance profiles of the set of control versus adenoma samples, the set of control versus carcinoma samples, and the set of adenoma versus carcinoma samples to generate their respective classification models; quantify the abundance of a sensory protein from the sequenced metagenomic reads using the database of sensory protein sequences; assess the risk of the person to be in the CRC diseased state using the respective classification models and the computed abundance of the sensory protein in the metagenomic sample of the person, wherein the assessment results in the categorization of the person either in a low risk, a medium risk or a high risk of colorectal cancer diseased state based on a predefined criteria; and provide a therapeutic construct to the person depending on the risk of the colorectal cancer.

In another aspect, a method for assessing the risk of colorectal cancer (CRC) in a person has been provided. Initially, a database of sensory protein sequences of a plurality of organisms is created, wherein the database of sensory protein sequences comprises information pertaining to the sensory proteins of all fully or partially sequenced bacterial genomes obtained from a plurality of public repositories. Further, sensory protein abundance profiles of a set of control versus adenoma samples, a set of control versus carcinoma samples, and a set of adenoma versus carcinoma samples obtained from publicly available data is generated. In the next step, a random forest classifier is applied on the generated sensory protein abundance profiles of the set of control versus adenoma samples, the set of control versus carcinoma samples, and the set of adenoma versus carcinoma samples to generate their respective classification models. Later, a microbiome sample is collected from a body site of the person for the assessment of the risk of CRC, wherein the microbiome sample comprising microbial cells. Later, DNA is extracted from the microbial cells. The extracted DNA is then sequenced via the sequencer to get sequenced metagenomic reads. In the next step, the abundance of a sensory protein is quantified from the sequenced metagenomic reads using the database of sensory protein sequences. Further, the risk of the person to be in the CRC diseased state is assessed using the respective classification models and the computed abundance of the sensory protein in the metagenomic sample of the person, wherein the assessment results in the categorization of the person either in a low risk, a medium risk or a high risk of colorectal cancer diseased state based on a predefined criteria. And finally, a therapeutic construct is provided to the person depending on the risk of the colorectal cancer.

In yet another aspect, one or more non-transitory machine readable information storage mediums comprising one or more instructions which when executed by one or more hardware processors cause assessing the risk of colorectal cancer (CRC) in a person. Initially, a database of sensory protein sequences of a plurality of organisms is created, wherein the database of sensory protein sequences comprises information pertaining to the sensory proteins of all fully or partially sequenced bacterial genomes obtained from a plurality of public repositories. Further, sensory protein abundance profiles of a set of control versus adenoma samples, a set of control versus carcinoma samples, and a set of adenoma versus carcinoma samples obtained from publicly available data is generated. In the next step, a random forest classifier is applied on the generated sensory protein abundance profiles of the set of control versus adenoma samples, the set of control versus carcinoma samples, and the set of adenoma versus carcinoma samples to generate their respective classification models. Later, a microbiome sample is collected from a body site of the person for the assessment of the risk of CRC, wherein the microbiome sample comprising microbial cells. Later, DNA is extracted from the microbial cells. The extracted DNA is then sequenced via the sequencer to get sequenced metagenomic reads. In the next step, the abundance of a sensory protein is quantified from the sequenced metagenomic reads using the database of sensory protein sequences. Further, the risk of the person to be in the CRC diseased state is assessed using the respective classification models and the computed abundance of the sensory protein in the metagenomic sample of the person, wherein the assessment results in the categorization of the person either in a low risk, a medium risk or a high risk of colorectal cancer diseased state based on a predefined criteria. And finally, a therapeutic construct is provided to the person depending on the risk of the colorectal cancer.

It is to be understood that both the foregoing general description and the following detailed description are exemplary and explanatory only and are not restrictive of the invention, as claimed.

### BRIEF DESCRIPTION OF THE DRAWINGS

The embodiments herein will be better understood from the following detailed description with reference to the drawings, in which:

FIG. 1 illustrates a block diagram of a system for assessing the risk of colorectal cancer in a person according to an embodiment of the present disclosure.

FIG. 2 shows a flowchart for creating a database of sensory protein abundances according to an embodiment of the disclosure.

FIG. 3 shows a workflow for the derivation of a ternary classification output based on binary classification according to an embodiment of the disclosure.

FIG. 4A-4B is a flowchart illustrating the steps involved in assessing the risk of colorectal cancer in the person according to an embodiment of the present disclosure.

FIG. 5 shows a block diagram for generating a classification model to be used in the system of FIG. 1 according to an embodiment of the disclosure.

### DETAILED DESCRIPTION OF EMBODIMENTS

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered as exemplary only, with the true scope being indicated by the following claims.

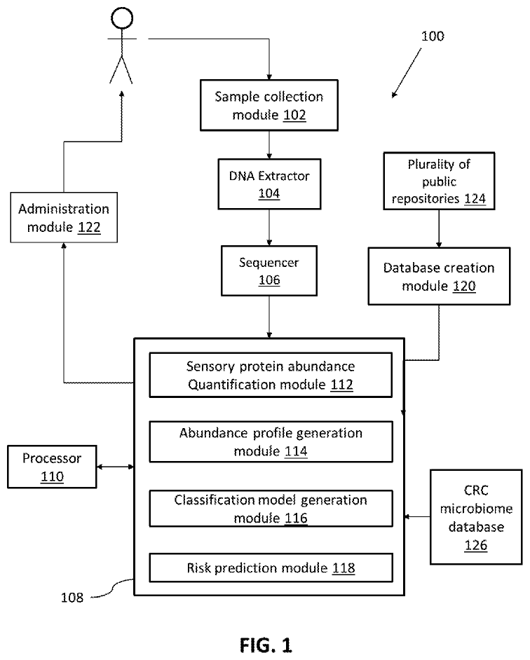

Referring now to the drawings, and more particularly to FIG. 1 through FIG. 5, where similar reference characters denote corresponding features consistently throughout the figures, there are shown preferred embodiments and these embodiments are described in the context of the following exemplary system and/or method.

According to an embodiment of the disclosure, a system 100 for assessing the risk of colorectal cancer in a person. The system 100 is configured to assess individuals to check the risk of presence of colorectal cancer (CRC) and/or adenomatous (colonic/rectal) polyps, by quantifying the abundance of sensory proteins in their gut microbiome. The system 100 further categorizes the person into one of healthy, adenoma and cancerous categories based on the nature and abundance of sensory proteins in the gut microbiome. The system 100 further describes microbiota based therapeutics for treatment of the person with colorectal adenoma and/or cancer through administration of at least one of a consortium of healthy microbes, antibiotic drugs and pre-/post-biotic compounds which could modulate the disease microbiome composition towards a healthy equilibrium.

According to an embodiment of the disclosure, the system 100 comprises of a sample collection module 102, a DNA extractor 104, a sequencer 106, a memory 108 and a processor 110 as shown in FIG. 1. The processor 110 is in communication with the memory 108. The processor 110 is configured to execute a plurality of algorithms stored in the memory 108. The memory 108 further includes a plurality of modules for performing various functions. The memory 108 may include a sensory protein abundance quantification module 112, an abundance profile generation module 114, a classification model generation module 116 and a risk prediction module 118. The system 100 also comprises a database creation module 120 using plurality of public repositories 124. The system 100 further comprises an administration module 122 as shown in the block diagram of FIG. 1. The system 100 also comprises a CRC microbiome database 126 as shown in the block diagram of FIG. 1.

According to an embodiment of the disclosure, the microbiome sample is collected using the sample collection module 102. The sample collection module 102 is configured to collect microbiome sample from gut of the person for the assessment of the risk of CRC, wherein the microbiome sample comprising microbial cells. The sample collection module 102 collect the microbiome sample in the form of saliva, stool, blood, or any other body fluids/swabs from at least one body site/location viz. gut, oral, skin etc. The microbiome sample can also be collected from subjects of different geographies. The microbiome sample can also be collected from one or multiple body sites at a single or longitudinal time points of healthy individuals or patients at various stages of CRC. The sample collection module 102 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite.

The system 100 further comprises the DNA extractor 104 and the sequencer 106. DNA is first extracted from the microbial cells constituting the microbiome sample using laboratory standardized protocols by employing the DNA extractor 104. Next, sequencing is performed using the sequencer 106 to obtain the sequenced metagenomic reads. The sequencer 106 performs whole genome shotgun (WGS) sequencing from the extracted microbial DNA, using a sequencing platform after performing suitable pre-processing steps (such as, sheering of samples, centrifugation, DNA separation, DNA fragmentation, DNA extraction and amplification, etc.) The extracted and sequenced DNA sequences are then provided to the processor 110.

In another embodiment of the disclosure, the DNA extractor 104 and sequencer 106 are also configured to use universal primers to kinase domains to specifically pull down and amplify DNA sequences fragments encoding for sensory kinases. They can also perform amplicon sequencing (such as, sequencing 16S rRNA gene, sequencing cpn60 gene, etc.) of the collected microbiome. Further, the DNA extractor 104 and the sequencer 106 are also configured to extract and sequence microbial transcriptomic (also referred to as meta-transcriptomic) data. The DNA extractor 104 and the sequencer 106 are also configured to perform any one of chip based hybridization, ELISA based separation, size/charge based seclusion of specific class of DNA/RNA/protein and subsequently perform amplification and sequencing and/or quantification of the same. Sequencing may be performed using approaches which involve either a fragment library or a mate-pair library or a paired-end library or a combination of the same. Sequencing may also be performed using any other approaches such as by recording changes in the electric current while passing a DNA/RNA molecule through a nano-pore while applying a constant electric field or by using mass spectrometric techniques.

According to an embodiment of the disclosure, the system 100 comprises the database creation module 120. The database creation module 120 is configured to create a database of sensory protein sequences of all the organisms, wherein the database of sensory protein sequences comprises information pertaining to the proteins of all fully sequenced bacteria obtained from a plurality of public repositories 124. The plurality of public repositories 124 may include, but not limited to NCBI, Protein Data Bank, KEGG, PFAM, EggNOG, etc. Thus, the database creation is a onetime process. The pre-created database of sensory protein sequences can be used for the diagnosis of CRC as explained in the later part of the disclosure.

In another embodiment of the disclosure, the database of sensory proteins created using the database creation module 120 may also include sensory protein sequences from partially sequenced bacteria and/or other microorganisms including but not restricted to viruses, fungi, micro-eukaryotes, etc. obtained from a plurality of public repositories 124. In another embodiment, the database creation module 120 is also configured to create the database of interactome proteins and create a database of any other types of protein group/functional class.

According to an embodiment of the disclosure, the memory 108 comprises the sensory protein abundance quantification module 112. The sensory protein abundance quantification module 112 is configured to compute the abundance of the sensory protein encoding genes in the sequenced metagenomic reads using the database of sensory protein sequences. In an embodiment, following methodology can be used to compute the sensory protein abundance for the sequenced metagenomic reads.

Step 1: Perform a sequence alignment such as tBLASTN with the sequences in the created sensory protein sequence database as query against the sequenced metagenomic reads. The hits satisfying a minimum e-value threshold of 1.0*e⁻⁵ (0.00001) were considered as correct matches.

Step 2: For each bacterial strain in the sensory protein sequence database the cumulative matches of the sequenced metagenomic reads are computed to form the “Count of sensors” which indicates approximately the potential number of sensory protein coding regions in the genome for that particular bacterial strain for the microbiome sample from which the sequenced metagenomic reads were obtained. Also for each bacterial strain in the sensory protein sequence database the cumulative length of the nucleotide bases for all these hits is computed to form the “Covered base length” which indicates approximately the total length of the potential sensory protein coding regions in the genome for that particular bacterial strain for the microbiome sample from which the sequenced metagenomic reads were obtained.

Step 3: The calculation of the sensory protein abundance can be performed using two implementations: In the first implementation, computation of sensory protein abundance is performed by calculation of the ratio of the “Count of sensors” to the total size of the sequenced metagenomic reads constituting the microbiome sample, henceforth referred to as metagenomic size (in Megabases). This ratio indicates the cumulative number of sensory proteins for that bacterial strain coded per unit of the sequenced metagenomic reads constituting the microbiome sample. Thus,

$$Sensory Protein Abundance = \frac{Count of Sensors for a particular strain}{Metagenomic Size}$$

In the second implementation, computation for the sensory protein abundance can be performed by calculation of the ratio of the “Covered base length” to the total metagenomic size (in Megabases) of the microbiome sample for each available bacterial strain. This ratio indicates the cumulative length of sensory protein coding regions (coding sequence) for that bacterial strain per unit of the sequenced metagenomic reads constituting the microbiome sample. Thus,

$$Sensory protein abundance = \frac{Covered base length for a particular strain}{Metagenomic Size}$$

The sensory protein abundance for the sequenced metagenomic reads can also be computed using various other implementations of the process and are described as follows. In one implementation, the computation can be performed at any of the known taxonomic levels or the computation can also be performed at each of the different taxonomic levels using a mixture of organisms. The sensory protein abundance is initially computed for each available strain(s) and in one implementation can be cumulated to a desired taxonomic level. In another implementations, the computed sensory protein abundance may be replaced by any other statistical means, such as mean, median, mode, etc. Organisms other than bacteria (either alone or in combination with other taxonomic lineages) may also be employed. In yet another implementation, one or more group of proteins, other than sensory proteins may be used, either alone or in combination with the sensory proteins and/or taxonomic classifications.

According to an embodiment of the disclosure, the memory 108 also comprises the abundance profile generation module 114, and the classification model generation module 116. The abundance profile generation module 114 is configured to generate sensory protein abundance profiles from sequenced metagenomic reads obtained from publicly available data. The set of sequenced metagenomic reads can be used for training and/or testing. The abundance profiles of the sequenced metagenomic reads is used as the training and/or testing data for the generation of a classification model and testing its efficiency. The classification model generation module 116 is configured to apply a random forest (RF) classifier on the sensory protein abundance profiles of the subset of sequenced metagenomic reads to generate a classification model and test prediction accuracy on the other subset. In one embodiment, the microbiome samples, constituting of sequenced microbiome reads may be obtained from publicly available CRC microbiome data through the CRC microbiome database 126. The microbiome samples, from which the sequenced metagenomic reads are obtained, are divided in a random set of 90% as the training set and rest of the 10% as the testing set. Thus, the generated classification model can also be used to classify the testing set as well.

According to an embodiment of the disclosure, the memory 108 comprises the risk prediction module 118. The risk prediction module 118 is configured to predict the risk of the person to be in the CRC diseased state using the generated classification model, wherein the prediction results in the categorization of the person either in a low risk, a medium risk or a high risk of colorectal cancer diseased state based on a predefined criteria. The risk prediction module 118 takes input from the sensory protein abundance quantification module 112. The machine learning technique of RF classifier was used for model based prediction using train and test set.

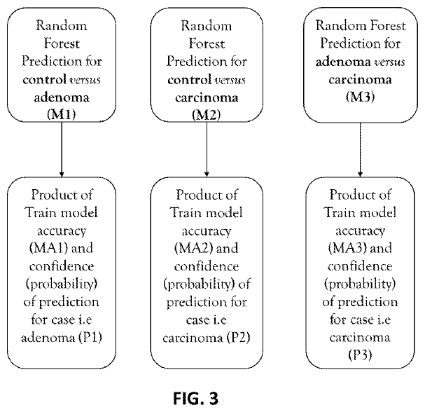

The classification model generation module 116 further creates three binary classification models, namely, control versus adenoma, control versus carcinoma, and adenoma versus carcinoma. However, these binary classification models cannot be directly used to infer on the ternary classification of a sequenced metagenomic reads obtained from the microbiome sample of the person being examined. The workflow for the derivation of a ternary classification output based on above mentioned binary classification models is shown in FIG. 3. TABLE 1 show the equations which were used to derive the ternary classification, where M1, M2 and M3 are Random Forest (RF) prediction for control vs adenoma, control vs carcinoma, and adenoma vs carcinoma respectively. MA1, MA2 and MA3 are the train model accuracies, P1, P2 and P3 are confidence (probability) of prediction for case of RF prediction for models control versus adenoma, control versus carcinoma, adenoma versus carcinoma respective to the model.

TABLE 1
Equations used to derive ternary classification

| | Control (A) | Adenoma (B) | 1 |
|---|---|---|---|
| | Prediction A | Prediction B | Prediction C |
| M1 | MA1*(1-P1) | MA1*P1 | 0 |
| M2 | 0 | MA2*(1-P2) | MA2*P2 |
| M3 | MA3*(1-P3) | 0 | MA3*P3 |
| Ternary | Sum of (M1, A), | Sum of (M1, B), | Sum of (M1, C), |
| Classification | (M2, A), (M3, A) | (M2, B), (M3, B) | (M2, C), (M3, C) |

The final risk prediction is based on the maximum score from the Ternary Classification i.e. if Prediction A is greater than Prediction B and Prediction C then the final prediction is A and the microbiome sample, comprising of sequenced metagenomic reads, would be predicted as Control. Similarly for the other cases microbiome sample, comprising of sequenced metagenomic reads, can be predicted as adenoma or carcinoma.

The predicted risk as explained above can be categorised into:

Prediction A: ‘Low risk (Apparently healthy)’

Prediction B: ‘Moderate risk (Adenoma/Polyps)’

Prediction C: ‘High risk (Carcinoma/Advanced Adenoma)’

In another embodiment of the disclosure, the following method can also be used to predict the diseased condition of the person based on sequenced metagenomic reads obtained from the microbiome sample. TABLE 2 shows the equation used to derive the ternary classification for predicting the risk (Prediction A: low risk; Prediction B: moderate risk Prediction A: high risk).

TABLE 2
A second set of equations used to derive ternary classification

| | Control (A) | Control (B) | Control (C) |
|---|---|---|---|
| | Prediction A | Prediction B | Prediction C |
| M1 | MA1*(1-P1) | MA1*P1 | MA1*P1 |
| M2 | MA2*P2 | MA2*(1-P2) | MA2*P2 |
| M3 | MA3*(1-P3) | MA3*P3 | MA3*P3 |
| Ternary | Sum of (M1, A), | Sum of (M1, B), | Sum of (M1, C), |
| Classification | (M2, A), (M3, A) | (M2, B), (M3, B) | (M2, C), (M3, C) |

Where M1, M2 and M3 are Random Forest (RF) prediction for control vs rest, adenoma vs rest, and carcinoma vs rest respectively. Further, while MA1, MA2 and MA3 are the train model accuracies, P1, P2 and P3 are probabilities of RF prediction for models control versus rest, adenoma versus rest, carcinoma versus rest respective to that model. Prediction shifts to the maximum from the Ternary Classification i.e. if Prediction A is greater than Prediction B and Prediction C then prediction shift is towards A and the microbiome sample, comprising of sequenced metagenomic reads, would be predicted as Control. Similarly for the other cases microbiome sample can be predicted as adenoma or carcinoma.

The predicted risk as explained above can be categorised into:

Prediction A: ‘Low risk (Apparently healthy)’

Prediction B: ‘Moderate risk (Adenoma/Polyps)’

Prediction C: ‘High risk (Carcinoma/Advanced Adenoma)’

According to another embodiment of the disclosure, RF prediction in two steps where in the first step is a binary classifier to predict the carcinoma samples and rest are then again subjected to another binary classification to predict between the adenoma and the control microbiome samples. In this technique no further equation is required to derive the ternary classification output but the binary classification is carried out at two levels as has been explained above. In alternate implementations, any of the classes may be removed/segregated/identified from the remaining two classes in the first binary classification step, and the remaining two classes may be further resolved in the second binary classification step. The use of any other machine learning/statistical approach as an alternate to RF for the binary classification step is well within the scope of this disclosure.

According to another embodiment of the disclosure, the ternary classification may be performed using multiclass classification techniques such as, neural networks, nearest neighbor approaches, naive Bayes, support vector machine, hierarchical classification, multidimensional scaling, principal component analysis, principal coordinates analysis, partial least squares discriminant analysis, gradient boosting algorithms, tree based classifiers etc.

According to an embodiment of the disclosure, the system 100 also comprises of the administration module 122. The administration module 122 is configured to provide/administer a therapeutic construct to the person depending on the risk of the colorectal cancer. It should be appreciated that any of the well-known technique can be used to administer the construct. The administration module 122 uses at least one of a consortium/construct of healthy microbes, antibiotic drugs and pre-/pro-/syn-/post-biotics or fecal microbiome transplant that would help the patient's gut microbiome to attain a healthy equilibrium without any adverse health effects. The therapy may be provided in the form of anyone (or a combination) of the known routes of administrations like intravenous solution, sprays, patches, band-aids, pills or syrup.

The therapeutics is suggested as a consortium of microbes based on their (inverse) correlation with the disease microbiome which can contribute to the therapeutic treatment for prediabetes by modulating the disease microbiome towards healthy equilibrium. Different implementations to identify the suitable therapeutic candidates are as following:
The sub-set of the reported screening markers abundant in healthy subjects, i.e. Healthy Therapeutic Markers (HTMs) which have been previously identified in research to be non-pathogenic
The different species and strains belonging to the same genus of the HTMs which have been previously identified in research to be non-pathogenic
All organisms having >90% identity and coverage over the genome of HTMs and which have been previously identified in research to be non-pathogenic
Any previously reported organisms which are known to boost the population of (non-pathogenic) HTMs and which have been previously identified in research to be non-toxic and do not cause any adverse effect
One or more of a natural or synthetically derived compounds which boost the population of (non-pathogenic) HTMs, wherein the natural or synthetically derived compounds are non-toxic
Any organism with identical sensory protein/kinase domain to HTMs and previously identified in research to be non-pathogenic/non-toxic
one or more of a natural or synthetically derived compounds which targets the reported screening markers abundant in diseased subjects, i.e. Disease Markers (DMs), wherein the natural or synthetically derived compounds are non-toxic and do not cause any adverse effect
Any organism previously reported, or any of its related similar organisms (similar through genomic make up or characteristic functions) which inhibit growth of reported screening markers abundant in diseased patients, i.e. Disease markers (DMs) and previously identified in research to be non-pathogenic.
Any sequence with above mentioned similarity to these sequences are also potential markers.

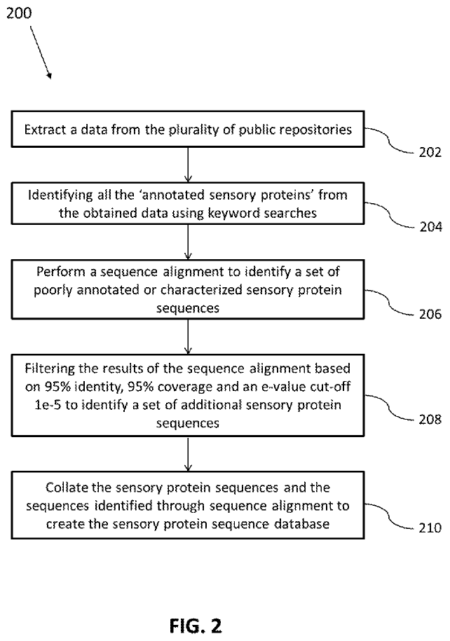

A flowchart 200 for creating a database of sensory protein sequence is shown in FIG. 2. Initially at step 202, a data is extracted from the plurality of public repositories 124. In the next step 204, all the ‘annotated sensory proteins’ from the obtained data were identified using keyword searches. At step 206, followed by a sequence alignment step (BLAST) to identify the poorly annotated/less characterized sensory protein sequences. For the purpose, the sequences corresponding to the ‘annotated sensory proteins’ were used as the database and the rest of the obtained bacterial protein sequences were used as query. At step 208, the results of the sequence alignment is filtered based on 95% identity, 95% coverage and an e-value cut-off 1.0*e⁻⁵ (0.00001) to identify a set of additional sensory protein sequences;

And finally, at step 210, the sensory protein sequences (those used as a database for the BLAST search) and the ones identified through BLAST analysis were collated into the sensory protein sequence database.

In another embodiment of the disclosure, the sequence alignment in step 206 may be performed using other techniques such as BLAT, DIAMOND, RAPSearch, BWA, Bowtie or through the use of clustering algorithms like BLASTCLUST, CLUSTALW, VSEARCH or any other heuristic techniques of identifying sequence similarity.

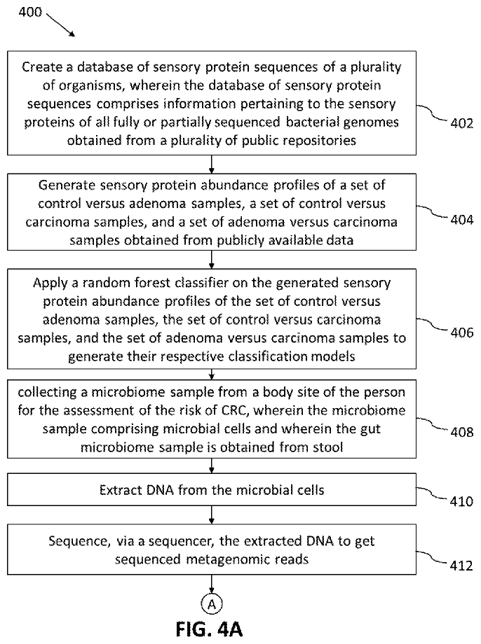

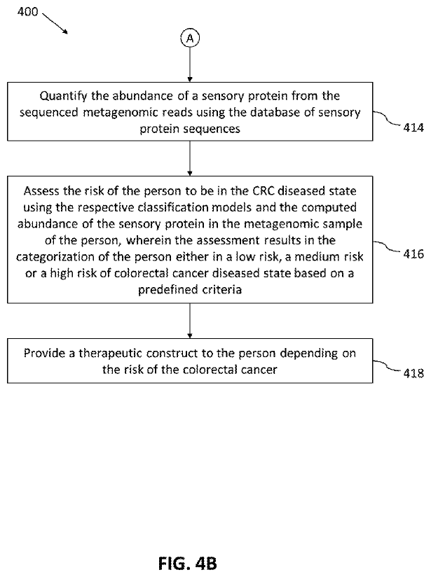

In operation, a flowchart 400 illustrating the steps involved for assessing the risk of colorectal cancer (CRC) in a person is shown in FIG. 4A-4B. Initially at step 402, a database of sensory protein sequences of a plurality of organisms is created. The database of sensory protein sequences created through database creation module 120 comprises information pertaining to the sensory proteins of all fully or partially sequenced bacterial genomes obtained from a plurality of public repositories 124. It may be appreciated that the database creation is a one-time process and created before the test sample from a person/patient is provided for the diagnosis and thereafter therapeutic purposes.

At step 404, the abundance profiles of a set of control versus adenoma samples, a set of control versus carcinoma samples, and a set of adenoma versus carcinoma samples obtained using the sensory protein abundance quantification module 112 and the abundance profile generation module 114 using data from the database creation module 120 utilizing publicly available repositories module 124. The set of samples constituting the publicly available data can be used for training or testing. The sensory protein abundance profiles of the samples are used as the training/testing data for the generation of the RF classification model using the classification model generation module 116. It may be appreciated that this generation of the classification model is a one-time process and created before the test sample from a person/patient is provided for the diagnosis and thereafter therapeutic purposes.

Further at step 406, the random forest classifier is applied on the generated sensory protein abundance profiles of the set of control versus adenoma samples, the set of control versus carcinoma samples, and the set of adenoma versus carcinoma samples to generate their respective classification models using the classification model generation module 116. It may be appreciated that this generation of the classification model is a one-time process and created before the test sample from a person/patient is provided for the diagnosis and thereafter therapeutic purposes.

At step 408, collecting a microbiome sample from gut of the person for the assessment of the risk of CRC, wherein the microbiome sample comprising microbial cells and wherein the gut microbiome sample is obtained from stool of the person. The gut microbiome sample, in the form of a stool sample, is collected from the person for the assessment of CRC. Though, it should be appreciated that the microbiome sample can also be collected from any other source. Further at 410, DNA is extracted from the microbial cells using DNA extractor 104. At step 412, the extracted DNA is sequenced via the sequencer 106 to get sequenced metagenomic reads.

At the next step 414, the abundance of a sensory protein from the sequenced metagenomic reads is quantified using the database of sensory protein sequences. At step 416, the risk of the person to be in the CRC diseased state is assessed using the respective classification models and the computed abundance of the sensory protein in the metagenomic sample of the person, wherein the assessment results in the categorization of the person either in a low risk, a medium risk or a high risk of colorectal cancer diseased state based on a predefined criteria. It may be noted that the CRC classification model was created using publicly available CRC microbiome data. It may be appreciated that this generation of the classification models is a one-time process and created before the test microbiome sample from a person/patient is provided for the diagnosis and thereafter therapeutic purposes. And finally at step 418, a therapeutic construct is provided to the person depending on the risk of the colorectal cancer using the administration module 122.

According to an embodiment of the disclosure, the system 100 for assessing the risk of the colorectal cancer in the person can also be explained with the help of following example. Publicly available gut microbiome data, comprising of sequenced metagenomic reads from stool microbiome samples, obtained from a previously published study was used for this evaluation. In this study, the number of gut microbiome samples, in the form of fecal/stool sample, corresponding to colorectal carcinoma, adenoma and healthy control are indicated below. There were a total of 155 microbiome samples, out of which 45 were stool microbiome samples from carcinoma patients, 47 were stool microbiome samples from adenoma patients and 63 were stool microbiome samples from healthy individuals and labelled as control samples. The sequenced metagenomic reads obtained from 155 shotgun-sequenced fecal/stool microbiome samples were used in the current evaluation and analysis.

A pairwise alignment using tBLASTN was performed using the derived sensory protein sequence database as query against the sequenced metagenomic reads. The protein-nucleotide translated BLAST, tBLASTN performs a comparison of a protein type query against all 6-frame translations of a nucleotide database. Blast hits satisfying the e-value threshold of 1.0*e⁻⁵ (0.00001) were used to calculate the sensory protein abundance across all bacterial strains, which constituted the sensory protein sequence database. For the current implementation the sensory protein abundance was calculated at species level. Sensory protein abundance was computed by cumulating the abundance of sensory proteins for all the bacterial strains, constituting the sensory protein sequence database, of a particular species for each of the fecal/stool microbiome samples.

State of the art machine learning technique was implemented for model based prediction of the samples as explained earlier. In order to implement the prediction methodology as a ternary classification technique, binary classification of control versus adenoma, control versus carcinoma and adenoma versus carcinoma were first performed. Then the inference of the binary classifications was used for ternary classification.

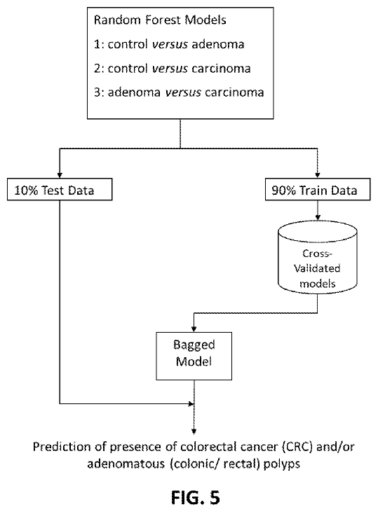

The Random Forest (RF) approach (R 3.0.2, randomForest4.6-7 package) was applied on the sensory protein abundance profiles of sequenced metagenomic reads as shown in the schematic block diagram of FIG. 5 (in alternate implementation other machine learning approaches such as XGBoost, neural networks, nearest neighbour approaches, naive Bayes, support vector machine, hierarchical classification, multidimensional scaling, principal component analysis, principal coordinates analysis, partial least squares-discriminant analysis, gradient boosting algorithms, tree based classifiers etc. may be used). A random set of sequenced metagenomic reads comprising 90% of the fecal/stool microbiome samples were selected as the training set and rest of the 10% were considered as the test set. Subsequently 10 replicates on 10-fold cross-validation were performed on the train dataset to build 100 cross-validation RF models (in alternate implementation, wherein no variable importance measures are employed, the cross-validation step may be avoided). The ‘importance’ of each of the features included in the cross-validation models was captured in form of GINI index (in alternate implementation, alternate forms of mean decrease of accuracy and/or mean decrease of impurity may be used in place of GINI index). ‘X’ most ‘important’ features (here X was equal to 10), based on GINI index values were selected from each of the 100 models (in alternate implementations, X may vary from 2 to ‘N’, wherein ‘N’ is the total number of features). Each feature in the sub-set of features, that was obtained by choosing the ‘X’ most ‘important’ features from each of the 100 cross-validation RF models, was subsequently ranked on the basis of the sum of their GINI index values (in alternate implementation, the features may be ranked on the basis of their occurrence frequency in the sub-set of features). Next, multiple ‘evaluation’ models were obtained by cumulatively adding the next ranked feature in the feature sub-set with the features of the previous ‘evaluation’ model, wherein the first ‘evaluation’ model comprised of the top two features in the feature sub-set. Subsequently, the performance of all the ‘evaluation’ models were assessed on the basis of their performance and the best performing ‘evaluation’ model was chosen as the final ‘bagged’ model. The performance of the ‘evaluation’ model was evaluated on the basis of Balancing Score, followed by Matthews correlation coefficient (MCC) and Area under the curve (AUC) scores. In cases where multiple models demonstrated identical performance measures, the ‘evaluation’ model with least number of features was chosen as the final ‘bagged’ model. The Balancing Score was computed as following.

Balancing Score=(sensitivity+specificity)−absolute(sensitivity−specificity)

The final ‘bagged’ model was then validated on the test set containing rest 10% of the dataset earlier kept aside as the independent test set. The accuracy of training model and the confidence probability of the prediction to be ‘case’ (control versus adenoma: case adenoma; control versus carcinoma: case carcinoma; adenoma versus carcinoma: case carcinoma) were accounted. This was further used for deriving the ternary classification.

In an embodiment of the disclosure, DNA fragments encoding for the set of kinase proteins which have been identified to be key differentiators between healthy, adenoma and CRC fecal/stool microbiome samples may be specifically measured using a PCR-based approach (such as, rtPCR, qPCR, etc.) or ELISA-based technique. In this case, primers specific to the proteins of interest may be designed to pull down the proteins of interest. This would enable for designing a CRC test kit which is highly affordable and can be used assessment of CRC risk among masses. This has been explained in detail in the later part of the disclosure. TABLE 3 below shows the results of cross validation. TABLE 4 provides a list of discriminating taxa (based on Sensory protein Abundance)

TABLE 3
Cross validation results on the train and the test data set

| | Train | | Test | |
|---|---|---|---|---|
| Classification Basis | Sensitivity | Specificity | Sensitivity | Specificity |
| Taxonomy (Genus)^{#} | 93.90 | 92.98 | 60.00 | 50.00 |
| Taxonomy (Species)^{#} | 90.24 | 92.98 | 60.00 | 50.00 |
| Sensory Proteins | 96.34 | 92.98 | 70.00 | 66.67 |
| Kinase proteins* | 95.12 | 91.23 | 70.00 | 66.67 |

^{#}Refer to results obtained using taxonomic abundances through 16S rRNA gene analysis. Taxonomic abundances were derived using C16S, an algorithm for taxonomic classification of 16S rRNA gene sequences from WGS metagenomic data.
*Refer to results obtained using an alternate implementation wherein a subset of proteins (those containing a kinase domain) in the sensory protein database is used as the backend database. Using this subset of proteins allow for preparing a test kit and a CRC screening protocol that is highly economical and can be easily deployed for mass CRC screening.

TABLE 4
List of discriminating taxa based on Sensory Protein
Abundances (SPAs). SPAs were calculated
using method explained earlier
without application of any other normalization techniques.

| Taxonomy | Healthy | Adenoma | Carcinoma |
|---|---|---|---|
| Bacillus anthracis | 787.158 | 743.884 | 576.889 |
| Bacillus infantis | 11.674 | 10.36 | 7.599 |
| Bartonella australis | 1.765 | 1.977 | 1.281 |
| Bartonella quintana | 3.518 | 3.984 | 2.586 |
| Bartonella tribocorum | 1.765 | 1.992 | 1.293 |
| Calothrix sp. | 40.12 | 40.211 | 30.149 |
| Candidatus saccharibacteria | 0.246 | 0.44 | 0.225 |
| Corynebacterium | 0.45 | 0 | 0.173 |
| kroppenstedtii | | | |
| Fibrobacter succinogenes | 86.196 | 77.134 | 41.987 |
| Haliangium ochraceum | 5.249 | 6.438 | 4.44 |
| Lactobacillus | 1.398 | 0.983 | 0.728 |
| sanfranciscensis | | | |
| Methanocaldococcus | 0.861 | 1.109 | 0.785 |
| infernus | | | |
| Nostoc punctiforme | 38.393 | 40.147 | 28.741 |
| Planctomyces limnophilus | 13.08 | 14.174 | 10.805 |
| Solitalea canadensis | 0.844 | 1.496 | 1.828 |
| Sphingobium | 3.292 | 4.19 | 3.097 |
| chlorophenolicum | | | |
| Stigmatella aurantiaca | 9.43 | 10.548 | 7.349 |
| Treponema caldaria | 12.122 | 12.142 | 7.576 |
| Veillonella parvula | 2.726 | 2.692 | 2.129 |

Based on the above results, one or more of the non-pathogenic HTMs, viz, Candidatus saccharibacteria, Fibrobacter succinogenes, Haliangium ochraceum, Calothrix sp., Lactobacillus sanfranciscensis, Methanocaldococcus infernus, Nostoc punctiforme, Planctomyces limnophilus, Sphingobium chlorophenolicum, Stigmatella aurantiaca, Veillonella parvula or other non-pathogenic organisms satisfying one or more of the above criteria may be considered as HTMs and administered either alone or in concoction for therapeutic purposes.

Alternatively, one or more pre-/pro-/syn-/post-biotics or fecal microbiome transplant may be used to boost the abundance/viability of HTMs, such as, Candidatus saccharibacteria, Fibrobacter succinogenes, Haliangium ochraceum, Calothrix sp., Lactobacillus sanfranciscensis, Methanocaldococcus infernus, Nostoc punctiforme, Planctomyces limnophilus, Sphingobium chlorophenolicum, Stigmatella aurantiaca, Veillonella parvula or other non-pathogenic organisms satisfying one or more of the above criteria may be administered either alone or in concoction for therapeutic purposes. Furthermore, antibiotic drugs may be administered to target Solitalea canadensis or any other organisms satisfying criteria for DMs. The proposed microbiome-based treatment may also be used in combination with one or more of traditional modes of treatment for CRC including low-dose chemotherapy, radiation therapy, etc.

Thus, the Random Forest (RF) model based prediction method can be efficiently applied to perform risk assessment of CRC, based on sensory protein abundance from the gut microbiome sample, which may be derived from the stool of an individual. In alternate implementations, microbiome samples may be collected from other body sites, such as (but not limited to) oral cavity, skin, nasopharynx, biopsy tissues, etc. The microbiome samples may be collected in the form of stool, blood, lavage, other body fluids, swab samples, etc. The sensory protein abundance profile of a microbiome sample is clearly a potential biomarker for prediction of diseased state. The disclosure provides a non-invasive and cost effective method as compared to the existing methods. The embodiments of present disclosure herein provides a method and system for assessing and treating colorectal cancer in the person.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein addresses unresolved problem of early assessment of colorectal cancer in the person. The embodiment provides a system and method to assess the risk of colorectal cancer (CRC) in a person. Further depending on the risk, the therapeutic construct is also provided.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation.

Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

1. A method for assessing the risk of colorectal cancer (CRC) in a person, the method comprising:
creating, via one or more hardware processors, a database of sensory protein sequences of a plurality of organisms, wherein the database of sensory protein sequences comprises information pertaining to the sensory proteins of all fully or partially sequenced bacterial genomes obtained from a plurality of public repositories, wherein the creating further comprises:
extracting a data from the plurality of public repositories,
identifying all annotated sensory proteins from the extracted data using a set of keyword searches,
performing a sequence alignment to identify a set of poorly annotated or characterized sensory protein sequences,
filtering the results of the sequence alignment based on 95% identity, 95% coverage and an e-value cut-off 1.0*e⁻⁵ (0.00001) to identify a set of additional sensory protein sequences, and
collating the sensory protein sequences and the sequences identified through sequence alignment to create the database of sensory protein sequences;
generating, via the one or more hardware processors, sensory protein abundance profiles of a set of control versus adenoma samples, a set of control versus carcinoma samples, and a set of adenoma versus carcinoma samples obtained from publicly available data;
applying, via the one or more hardware processors, a random forest classifier on the generated sensory protein abundance profiles of the set of control versus adenoma samples, the set of control versus carcinoma samples, and the set of adenoma versus carcinoma samples to generate their respective classification models;
collecting a microbiome sample from a body site of the person for the assessment of the risk of CRC, wherein the microbiome sample comprising microbial cells;
extracting DNA from the microbial cells;
sequencing, via a sequencer, using the extracted DNA to get sequenced metagenomic reads;
quantifying, via the one or more hardware processors, the abundance of a sensory protein from the sequenced metagenomic reads using the database of sensory protein sequences;
assessing, via the one or more hardware processors, the risk of the person to be in the CRC diseased state using the respective classification models and the computed abundance of the sensory protein in the metagenomic sample of the person, wherein the assessment results in the categorization of the person either in a low risk, a medium risk or a high risk of colorectal cancer diseased state based on a predefined criteria; and
providing a therapeutic construct to the person depending on the risk of the colorectal cancer.

2. The method of claim 1, wherein the therapeutic construct comprises one or more non-pathogenic Healthy Therapeutic Markers (HTMs), a plurality of antibiotic drugs targeted against Disease Markers, pre-/pro-/syn-/post-biotics or fecal microbiome transplant to help the person's gut microbiome to attain a healthy equilibrium.

3. The method according to claim 1, wherein, the therapeutic construct comprises one or more of:
a plurality of Healthy Therapeutic Markers (HTMs), wherein the plurality of Healthy Therapeutic Markers are non-pathogenic,
species and strains belonging to same genus of the HTMs, wherein the species and strains are non-pathogenic,
a plurality of organisms having more than 90 percent identity and coverage over the genome of HTMs, wherein the plurality of organisms are non-pathogenic,
one or more organisms which boost the population of HTMs, wherein the one or more organisms are non-pathogenic, or
one or more of a natural or synthetically derived compounds which boost the population of HTMs, wherein the natural or synthetically derived compounds are non-toxic.
one or more of a natural or synthetically derived compounds which target the Disease Markers (DMs), wherein the natural or synthetically derived compounds are non-toxic and do not cause any adverse effects.

4. The method according to claim 3, wherein the plurality of Healthy Therapeutic Markers (HTMs) comprises one or more of Candidatus saccharibacteria, Fibrobacter succinogenes, Haliangium ochraceum, Calothrix sp., Lactobacillus sanfranciscensis, Methanocaldococcus infernus, Nostoc punctiforme, Planctomyces limnophilus, Sphingobium chlorophenolicum, Stigmatella aurantiaca, or Veillonella parvula, and administered either alone or in concoction for therapeutic purposes.

5. The method according to claim 3, wherein the Disease Marker (DM) comprises Solitalea canadensis.

6. The method according to claim 1, wherein the step of assessing the risk is based on a maximum score from a ternary classification, wherein the ternary classification is derived using outputs of the respective binary classification models based on a predefined condition.

7. The method according to claim 1, wherein the sample is collected in the form of one or more of saliva, stool, blood, body fluids, or swabs from at least one body site of the person, wherein the body site comprising one or more of gut, oral, or skin of the person.

8. (canceled)

9. The method according to claim 1, wherein the sequence alignment is performed using one or more of Basic Local Alignment Search Tool (BLAST), BLAST-like alignment tool (BLAT), DIAMOND alignment tool, RAPSearch tool, Burrows-Wheeler Aligner (BWA), Bowtie or through the use of clustering algorithms comprising BLASTCLUST, CLUSTALW, VSEARCH or heuristic techniques of identifying sequence similarity.

10. The method according to claim 1, wherein the plurality of public repositories comprises one or more of NCBI database, Protein Data Bank, KEGG database, PFAM database or EggNOG.

11. The method according to claim 1, wherein the step of generating classification models comprises:
applying a Random Forest (RF) approach on the sensory protein abundance profiles of sequenced metagenomic reads;
selecting a random set of sequenced metagenomic reads comprising 90% of the fecal/stool microbiome samples as a training set and rest of the 10% were considered as a test set;
performing 10 replicates on 10-fold cross-validation on the training set to build 100 cross-validation RF models;
capturing an importance of each of the features included in cross-validation models in terms of GINI index;
selecting a predefined number of most ‘important’ features based on GINI index values from each of the 100 cross-validation RF models to obtain a feature sub-set;
ranking each of the features in the feature sub-set, on the basis of the sum of their GINI index values;
obtaining multiple evaluation models by cumulatively adding the next ranked feature in a sub-set of features with the features of the previous ‘evaluation’ model, wherein the first ‘evaluation’ model comprised of the top two features in the feature sub-set;
assessing the performance of all the ‘evaluation’ models on the basis of their added features;
choosing the best performing ‘evaluation’ model as the final classification model; and
evaluating the performance of the ‘evaluation’ model on the basis of a balancing Score, followed by Matthews correlation coefficient (MCC) and Area under the curve (AUC) scores;
validating the final classification model on the test set containing rest 10% of the dataset earlier kept aside as the independent test set, wherein the accuracy of a training model and the confidence probability of the prediction to be ‘case’ (control versus adenoma: case adenoma; control versus carcinoma: case carcinoma; adenoma versus carcinoma: case carcinoma) were accounted.

12. The method according to claim 1, further comprising calculating the abundance of the sensory protein, comprises:
performing a sequence alignment with the sequences in the created sensory protein sequence database as query against the sequenced metagenomic reads, wherein the hits satisfying a minimum e-value threshold of 1.0*e⁻⁵ (0.00001) are considered as correct matches;
computing the cumulative matches of the sequenced metagenomic reads to form a count of sensors for each bacterial strain in the sensory protein sequence database, wherein the count of sensors indicates approximately the potential number of sensory protein coding regions in the genome for that particular bacterial strain for the microbiome sample from which the sequenced metagenomic reads were obtained;
computing the cumulative length of the nucleotide bases for all these hits for each bacterial strain in the sensory protein sequence database to form a covered base length, wherein the covered base length indicates approximately the total length of the potential sensory protein coding regions in the genome for that particular bacterial strain for the microbiome sample from which the sequenced metagenomic reads were obtained;
calculating the sensory protein abundance using one of the following:
calculating ratio of the count of sensors to the total metagenomic size (in Megabases) wherein total metagenomic size (in Megabases) is the size of the sequenced metagenomic reads constituting the microbiome sample, or
calculating the ratio of the covered base length of the particular strain to the total metagenomic size (in Megabases) of the microbiome sample for each available bacterial strain.

13. A system for assessing the risk of colorectal cancer in a person, the system comprises:
a sample collection module for collecting a microbiome sample from gut of the person for the assessment of the risk of CRC, wherein the microbiome sample comprising microbial cells;
a DNA extractor for extracting DNA from the microbial cells;
a sequencer for sequencing the extracted DNA to get sequenced metagenomic reads;
a database creation module for creating a database of sensory protein sequences of a plurality of organisms, wherein the database of sensory protein sequences comprises information pertaining to the proteins of all fully and partially sequenced bacterial genome obtained from a plurality of public repositories, wherein the database creation module further configured to:
extract a data from the plurality of public repositories,
identify all annotated sensory proteins from the extracted data using a set of keyword searches,
perform a sequence alignment to identify a set of poorly annotated or characterized sensory protein sequences,
filter the results of the sequence alignment based on 95% identity, 95% coverage and an e-value cut-off 1.0*e⁻⁵ (0.00001) to identify a set of additional sensory protein sequences, and
collate the sensory protein sequences and the sequences identified through sequence alignment to create the database of sensory protein sequences;
one or more hardware processors;
a memory in communication with the one or more hardware processors, wherein the one or more first hardware processors are configured to execute programmed instructions stored in the memory, to:
generate sensory protein abundance profiles of a set of control versus adenoma samples, a set of control versus carcinoma samples, and a set of adenoma versus carcinoma samples obtained from publicly available data;
apply a random forest classifier on the generated sensory protein abundance profiles of the set of control versus adenoma samples, the set of control versus carcinoma samples, and the set of adenoma versus carcinoma samples to generate their respective classification models;
quantify the abundance of a sensory protein from the sequenced metagenomic reads using the database of sensory protein sequences;
assess the risk of the person to be in the CRC diseased state using the respective classification models and the computed abundance of the sensory protein in the metagenomic sample of the person, wherein the assessment results in the categorization of the person either in a low risk, a medium risk or a high risk of colorectal cancer diseased state based on a predefined criteria; and
provide a therapeutic construct to the person depending on the risk of the colorectal cancer.

14. A computer program product comprising a non-transitory computer readable medium having a computer readable program embodied therein, wherein the computer readable program, when executed on a computing device, causes the computing device to:
create a database of sensory protein sequences of a plurality of organisms, wherein the database of sensory protein sequences comprises information pertaining to the sensory proteins of all fully or partially sequenced bacterial genomes obtained from a plurality of public repositories, wherein the creating further comprises:
extracting a data from the plurality of public repositories,
identifying all annotated sensory proteins from the extracted data using a set of keyword searches,
performing a sequence alignment to identify a set of poorly annotated or characterized sensory protein sequences,
filtering the results of the sequence alignment based on 95% identity, 95% coverage and an e-value cut-off 1.0*e⁻⁵ (0.00001) to identify a set of additional sensory protein sequences, and
collating the sensory protein sequences and the sequences identified through sequence alignment to create the database of sensory protein sequences;
generate sensory protein abundance profiles of a set of control versus adenoma samples, a set of control versus carcinoma samples, and a set of adenoma versus carcinoma samples obtained from publicly available data;
apply a random forest classifier on the generated sensory protein abundance profiles of the set of control versus adenoma samples, the set of control versus carcinoma samples, and the set of adenoma versus carcinoma samples to generate their respective classification models;
collect a microbiome sample from a body site of the person for the assessment of the risk of CRC, wherein the microbiome sample comprising microbial cells;
extract DNA from the microbial cells;
sequence, via a sequencer, using the extracted DNA to get sequenced metagenomic reads;
quantify the abundance of a sensory protein from the sequenced metagenomic reads using the database of sensory protein sequences;
assess the risk of the person to be in the CRC diseased state using the respective classification models and the computed abundance of the sensory protein in the metagenomic sample of the person, wherein the assessment results in the categorization of the person either in a low risk, a medium risk or a high risk of colorectal cancer diseased state based on a predefined criteria; and
provide a therapeutic construct to the person depending on the risk of the colorectal cancer.
